(19) United States
(12) Patent Application Publication
(10) Pub. No.: US 2010/0129909 A1
(43) Pub. Date: May 27, 2010

(54) DEVICE AND METHOD FOR TREATING BIOMASS

(76) Inventor: Earnest Stuart, Lansing, MI (US)

Correspondence Address: FURR LAW FIRM, 2622 DEBOLT ROAD, UTICA, OH, 43080, US

(21) Appl. No.: 12/445,285

(22) PCT Filed: Oct. 11, 2006
(86) PCT No.: PCT/US06/39603

§ 371 Date: Dec. 14, 2009

Related U.S. Application Data
(60) Provisional application No. 60/725,332, filed on Oct. 11, 2005.

(51) Int. Cl. C12N 5/04; C12N 5/07; C12M 1/00

(52) U.S. Cl. 435/375; 435/410; 435/283.1

(57) ABSTRACT

A method for pretreating and disrupting cell structure of biomass by subjecting the biomass to high pressure, thereby disrupting the cell structure of the biomass is provided. Also provided is a device for performing the method, wherein the device includes a cavitating device and a cell structure disrupting device disposed within the cavitating device for disrupting the cell structure and exposing the internal cell structure to enzymes.

### BACKGROUND OF THE INVENTION

1. Field of the Invention

Generally, the present invention relates to a method and devices for treating and refining biomass or for creating an advanced ruminant animal feed. More specifically, the present invention relates to disrupting the cellular structure of biomass while chemically hydrolyzing portions of the biomass, and rendering biomass more amenable to enzymatic hydrolysis, digestion, and gasification, while minimizing treatment times and chemical loadings for chemical hydrolysis, and producing higher quality products.

2. Description of the Related Art

As used herein, the term “biomass” includes any organic matter (whole, fractions thereof, and/or any components thereof) available on a renewable) basis, such as dedicated energy crops and trees, agricultural food and feed crops, agricultural crop wastes and residues, wood wastes and residues, aquatic plants, animal wastes, municipal wastes, and other waste materials. Additionally raw materials include, but are not limited to, cellulose-containing materials, native or treated, such as corn-fiber, hay, sugar cane bagasse, starch-containing cellulosic material such as grain, crop residues, newsprint, paper, raw sewage, aquatic plants, sawdust, yard wastes, grass, biomass, including by not limited to pretreated biomass, components thereof, fractions thereof, and any other raw materials or biomass materials known to those of skill in the art. Lignocellulose-containing fiber, and in the case of grains, includes starch, herein referred to as “biomass”, can be refined into sugars, protein, and lignin, and chemicals for gasification into methane or hydrogen production. The market for sugars, including xylose, arabinose, fats, oils, lignin, as well as glucose from the cellulosic portions of biomass, is in the tens of billions of dollars per annum, and may ultimately rise to as high as $100-200 billion per annum world wide as oil supplies dwindle and other factors affect existing fuel supply. With oil prices rising with the potential to rise even further, the demand for an alternative to gasoline and diesel is growing.

Biomass structures are naturally resistant to penetration by low levels of chemicals and/or process heat transfer, or to enzymatic hydrolysis. Reducing native biomass to extremely fine particle sizes, and further disrupting those ultra-fine biomass particles by blowing out their structures creates vast surface area, inside and outside, which allows more intimate penetration of hot liquids, chemicals, heat and/or enzymes to greatly enhance dissolving reactions and in producing more reactive biomass while minimizing those inputs. The only physical way to reach such levels, in part, has been with ultra fine mechanical grinding of dry biomass, or extreme application of cavitation with inline homogenizers, and to a lesser degree of effectiveness, by use of steam explosion. The levels of biomass destruction required to provide a highly reactive substrate using dry grinding is tremendously expensive, and does little to blow out the walls of the remaining fibrous structure, thus limiting bioreactivity and heat transfer in processes employing heat as a dissolving mechanism.

Concentrated acid, dilute, high-temperature acid combinations, steam, moderate temperature, neutral pH, dry grinding, strong alkali, liquid anhydrous ammonia, high water ratios of lime, conically-shaped rotor-stator tools, a laboratory sonicating device, liquid stream, high-shear, and cavitating devices have been used to attempt to refine biomass economically. Cavitation with inline homogenizers without other inputs requires too many repeated applications for practical biomass refining. Presently, there are no economical industrial-scale processes in operation for converting high percentages of native, non-starch biomass, especially the cellulosic portions, into organic acids, glucose, xylose or ethanol, or effectively into rumen animal feed without creating significant waste streams, or which result in a high percentage conversion of the biomass, or can achieve refining without requiring costly separation methods. It would therefore be useful to develop a method and device to more effectively and efficiently treat and dissolve both hemicellulosic and cellulosic portions of biomass.

### SUMMARY OF THE INVENTION

According to the present invention, there is provided a method for pretreating, disrupting the cellular structures of biomass, chemically hydrolyzing portions of it, and preparing primarily the remaining cellulosic portion for enzymatic hydrolysis by subjecting the biomass to rapid pressure changes combined with acidic or alkaline pH conditions at a wide range of high temperatures for short residence times, thereby disrupting the cell structure of the biomass without creating excessive fermentation inhibitors, minimizing waste streams, and while providing a substrate requiring more practical enzyme concentrations for hydrolysis. Also provided is a device or devices and parameters for use of device or devices for performing the method, wherein the device includes a cavitating and cell structure disrupting device disposed within the cavitating device for disrupting the cell structure and exposing the internal cell structure to enzymes. The present invention provides biomass particles with extreme surface area compared to other methods, and does so in a significantly more cost effective way.

### BRIEF DESCRIPTION OF THE DRAWINGS

Other advantages of the present invention are readily appreciated as the same becomes better understood by reference to the following detailed description, when considered in connection with the accompanying drawings wherein:

FIG. 1 is a photograph of a cell;

FIG. 2 is a photograph showing a disrupted cell wall; and

FIG. 3A is a graph showing corn fiber hydrolysis; and

FIG. 3B is a table showing the data depicted in the graph of FIG. 3A.

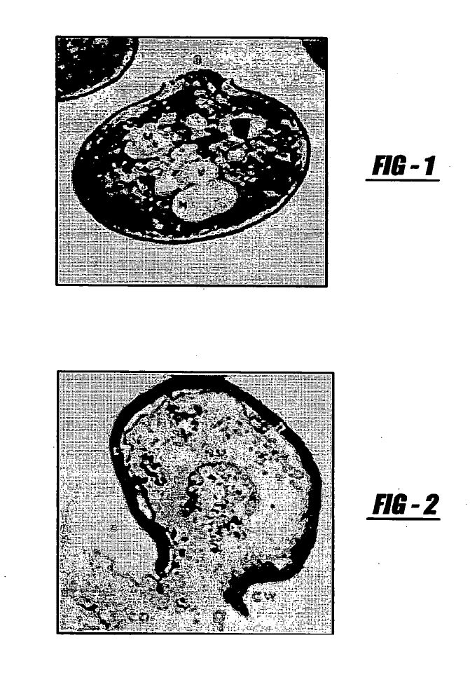

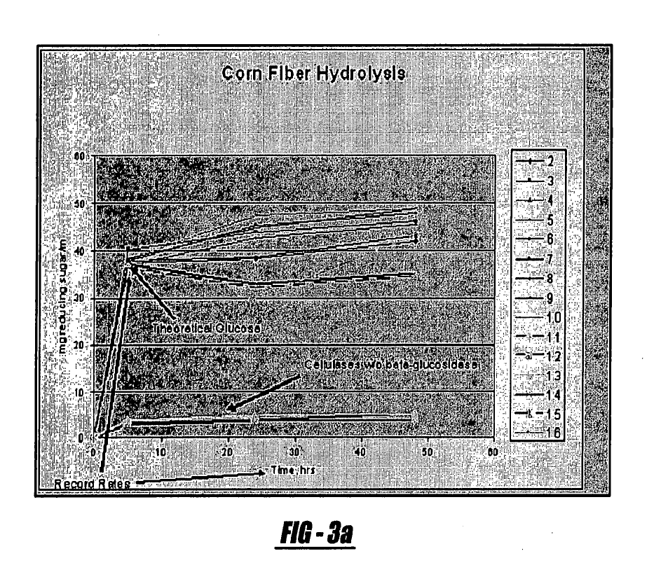

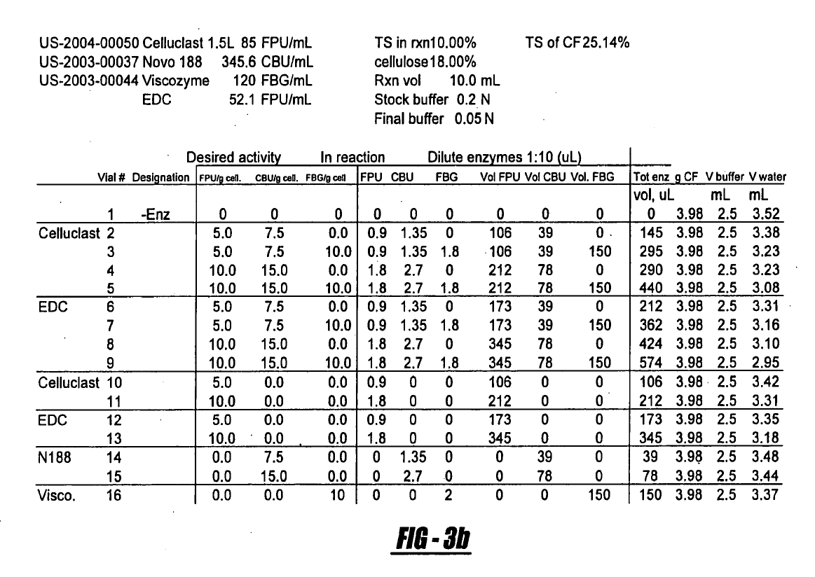

### DETAILED DESCRIPTION OF THE INVENTION

Generally, the present invention provides a method for processing and disrupting the primary cell structure of biomass, and chemically dissolving, a major component of biomass followed by dissolving most remaining components with low enzyme loadings with rapid dissolving rates. The cellular structure can be disrupted by subjecting the biomass to acidic or alkaline chemicals and related pH conditions with the addition of a wide range of heat, combined with high shear and cavitation under a range of equipment tip speeds and pressures, induced under a wide range of elevated pressures at the entrance of specially designed openings, and low exiting pressure zones within systems, followed by high pressure zones created by moving rotors. In some applications, chemical concentrations and corresponding pH conditions can be altered, either to become more basic or more acidic, during or after the application of cavitation, which has been applied with the addition of various levels of heat resulting in a wide range of temperatures. The method includes dissolving parts of the biomass with mild chemical conditions without enzymes, and provides the basis for refining biomass into its primary components of sugars, proteins and lignin, and their downstream products including, but not limited to, ethanol, sugar alcohols, organic acids, methane and other gases, milk and beef, and other commodities for chemical and hydrogen production.

The present invention also provides devices, mechanical operating parameters within devices, chemicals, chemical concentrations, pH conditions, pressures, higher temperatures and residence times for performing the method described above, wherein the devices include liquid stream, high-shear and cavitating devices and cell structure disrupting devices within the high shear and cavitating devices for disrupting the cell structure and exposing valuable components within the cell to dissolving enzymes, operated at various ranges of conditions and configurations depending upon substrate and target rates and yields of hydrolysis for commercial purposes. The method can include pretreating the biomass with high shear and cavitation to temperatures up to a boiling point of the water in the biomass without boiling the water in the biomass, for example up to 100 degrees centrigrade. Additionally, the present invention can utilize high temperature, in excess of 150 degrees Celsius, during the hydrolyzing, cavitating, and shearing step without forming as much of the degradation byproducts as found in the prior art methods. Further, such temperatures enable the use of nitric acid, as opposed to sulfuric acid as is used in the prior art methods and devices.

The phrases “cell disrupting device”, high-shear device, or cavitation device as used herein are intended to refer to a device capable of disrupting the cell wall/membrane under a wider range of pH and pressure conditions, temperatures and residence times. In other words, under controlled conditions, the device of the present invention can disrupt the cell wall/membrane and provide cellulase type enzymes access into the cell, or cellulase type enzymes in the stomach of a cow or other ruminant animals. The device can also provide for an acid hydrolysis of portions of the biomass while creating little to no fermentation inhibitors.

The cell structure-disrupting device can be a single orifice with the slurry driven by a high-pressure pump, or a tooth and chamber tool in a rotor-stator device containing many high-pressure passageways of various shapes including square, rectangular or other shapes, or a number of round nozzle holes within a rotor-stator device.

A pump-fed single nozzle tool can operate at a wide range of pressures and orifice sizes. Single high-pressure nozzles that can be used in the present method can reach pressures of 10,000 PSI when driven by staged progressive-cavity pumps.

In a rotor-stator device, the slurry is forced by the rotor through a series of coaxial meshing rings manufactured with slots or round holes. The rings, configured with teeth, are generally known as tooth and chamber tools and those configured with bore holes are generally known as nozzle tools. Nozzle holes in related commercial machines typically impose a higher energy at the point of work, specifically at the exit or downstream outlet of the holes, as compared to energy imposed at the slurry exit of a tooth and chamber type tool. By example, at the point of work on the downside of a gap in one brand of device, a Cavitron, containing tooth and chamber “tools” in which the gap is 2 mm, the shear energy at the point of work is 2×105. By comparison, the shear energy at the point of work of a 2 mm nozzle tool in the same machine is 5×107. Generally, the tooth and chamber tools cause high-shear whereas the nozzle tools induce high-shear and high-vortex cavitation. A certain degree of cellular disruption can occur within the tooth and chamber, but the nozzle tool induces the maximum pressure and release of pressure, cavitation.

Generally, in rotor-stator devices operating at much lower pressures than described for high pressure progressive cavity pumps, multiple tooth and chamber tools can be attached concentrically to the rotor and to the stator when tooth and chamber tools are used. Gaps between the “teeth” can vary in size. When nozzle tools are used, generally, a tooth and chamber type tool is affixed to the inner stage of the rotor, with one, two, or more outer rotor rings consisting of nozzle tools and a nozzle tool can be affixed to the stator on all stages of a multi-stage device. Tooth and chamber and other non-round holes are used for coarse breakdown of biomass rather than the higher shear or cavitation imposed by the smaller opening surface area of typical nozzle designs, mainly because the slightly larger particles can go through a tooth and chamber tool easier without clogging the device, than through a nozzle tool, and a nozzle tool focuses energy more efficiently than a slot, a larger square hole or similar shapes. The space between the rotor and stator of both types of tool configurations is typically about 1 mm, regardless of the tool, but this can vary. Even the first stage within a tooth and chamber tool can reduce particle size below 1 mm, or at least the width of the particle, while the length may be longer as the particle moves parallel to the rotor's direction of movement. For this reason, the use of multiple concentric rings is preferred to the use of a single stage for processing biomass as much size reduction and standardization of size can occur with multiple rings. However, a single stage device can be used in many instances, depending upon substrate, in combination with other parameters described herein, to affect extreme particle size reduction and creation of internal surface area. In many instances such a device can cost less to manufacture and replace internal components that wear rapidly. A single stage device on many substrates can be a cost effective tool within this process for achieving high levels of hydrolysis. Cavitation conditions can be impacted by entry-side, and exit-side pressures of the tooth and chamber, under certain conditions, or in nozzle tools. These factors include, but are not limited to, horsepower of motor or pump, tip speed, tool diameter, viscosity, etc. The viscosity of the biomass can also be altered to adjust the cavitation of the biomass. The viscosity is not limited to specifics as in the prior art methods. Instead the viscosity is only limited by the ability of the biomass to pass through the device of the present invention at rates relatively close to water-only flow design standards, for and while inducing maximum shear and cavitation. In a rotor-stator device, as the high-pressure slurry enters the controlled-shape passageway, such as a round orifice as one example, velocity increases as the slurry passes through the orifice. Then, the pressure of the slurry containing the biomass exceeds the vapor pressure of the slurry at the exit of the orifice, causing a violent expansion of the liquid inside and adjacent to the biomass, most of which is vaporized, thus creating high collapsing pressure. More specifically, a high speed jet coming out of an opening generates large velocity gradient between the jet and the ambient liquid. The large velocity gradient generates a strong vortex field and shear stress field. Low pressure is generated at the center of a vortex. The stronger the vortex the lower the pressure generated. When the pressure is below the vapor pressure of the liquid, the liquid evaporates to generate cavitation bubbles. When the cavitation bubble is carried to where pressure is higher than the vapor pressure, the bubble collapses to become liquid again. The rapid vaporization and condensation process is called cavitation. Extremely high impact pressure is generated at the final stage of collapse due to liquid surface colliding with liquid surface. Due to asymmetry of the flow field, a bubble usually does not collapse in spherical form. It has been observed that a high speed micro jet of supersonic speed can occur and generate extremely high pressure and temperature of short duration when the micro jet strikes a liquid surface or a solid surface. The high pressure, rather than the shear stress, is responsible for damaging of the nearby material. Cavitation is more likely to occur when jet velocity is higher and when there are gas nuclei present. Therefore, a device with many small size openings generates more cavitation bubbles and, hence, is more efficient. Within the method of the present invention, a slurry exiting the nozzle encounters a vacuum created by a passing rotor traveling at 150 feet per second, or more, or in some cases, less. Following such a condition, an equally powerful compressive force collapses the bubble created. This complete sequence is cavitation and exerts tremendous stress on biomass cells contained within the slurry, in part due to the liquid inside the cells that expand during the first phase of cavitation.

The right conditions of pressure drop, pH, and temperature on a given biomass substrate results in disruption of the cell's structure upon exiting the slurry passageway, while minimizing degradation products. In most devices imposing such conditions, an equally violent recompression of the water vapors into liquid and upon the embedded biomass, causes even further cellular and gross structure destruction of the biomass. It is said that the internal temperature of such “bubbles” reaches 5000 degrees for a fraction of a second. The shock wave of the recompression is very intense, and is known to destroy propellers on ships over time. The cell structure disrupting device is capable, if desired, of increasing the pressure on the entry to the nozzle or other shaped passageway and correspondingly the embedded biomass cells in elevated temperature, acidic conditions or high pH and heat swollen conditions, as an example, by increasing the speed of a slurry feed pump, or the shaft speed and correspondingly, the feet per second rate of a rotor, or “tip speed”, as well as by increasing the diameter of the various rings. In certain nozzle devices, exit pressure can be dropped further as well.

The term “tip speed” in describing the workings in a rotor-stator device is defined as the rate at which a point on the rotor, of a rotor-stator device, passes a fixed point on the corresponding stator, if that pathway was laid out in a direct line and measured by feet or meters. Typical speeds for many commercial, lower-speed, high-shear cavitation devices are approximately 50 feet per second, and as low as 40 feet per second. Even lower tip speeds occur in the inner rings of multi-staged devices wherein the tools are concentric and are ever larger while still attached on the same plane. Higher speed cavitation devices presently available with nozzle tools can have a tip speed of 70-160 feet per second, or higher tip speeds in newer designs on the drawing board. The tip speed and hole must be sized to the types of biomass to be successfully treated and relates to the viscosity, entry particle size and solids loadings possible within a pumpable slurry. Preferably, the tip speed of the device is at least 51 feet per second. It is preferred that the tip speed be at least 100 feet per second and in the preferred embodiment the tip speed is at least 150 feet per second. The slower speed devices typically cannot pass biomass through the 1.5-2 mm holes when the slurry contains even low solids loadings of 2.5%, unless the biomass has been hammermilled or other type milling to extremely fine particle sizes. Often, a tooth and chamber type device is used to prepare a slurried biomass for passage through the typically smaller nozzle orifice devices. Higher tip speeds are required when nozzle holes are in the 1.5 mm-2 mm diameter size, in order to have sufficient pressure to force the slurried biomass through the orifice. Presently only a few machines meet such a standard, including but not limited to the Supraton and the Cavitron, which are essentially the same design in the internal working components, both of which can operate at approximately 150 feet per second of tip speed. It is important to note while two specific machines are referenced herein, any machine capable of performing the requisite functions set forth herein can be utilized without departing from the spirit of the present invention. Further, slower machines of the same type can potentially process biomass in a similar way, but the faster the machine, the higher percentage of solids that can be processed, contributing to a more economical process.

The combinations of rotor-stator speed, shaft speed, entry pressure, pressure drop, tooth and chamber and nozzle tools, nozzle-nozzle tools, gap and hole sizes of each tool, number of tool sets in a given machine, rate of slurry flow, particle size of biomass, solids-loadings of biomass, percentage of silica, type of biomass including different lignin percentages, temperature of slurry, residence time at elevated temperatures, number of passes through any combination of above parameters, special engineered shapes of each of the above tools, special wear designs to extend life of tools, pH conditions, chemical concentration, etc., can all be synthesized in a wide number of configurations to produce an optimized pretreatment of a given type of biomass. All possible combinations can be adjusted to produce a wide range of optimal final hydrolysis rates and yields of hydrolyzed sugars, proteins, separated lignin and minerals, ratios of cellulolytic enzymes to biomass, combinations of other types of enzymes, additives to enhance rates of hydrolysis, and methods of recycling cellulase enzymes, or to create formulated, highly digestible cattle feed.

The wide range of parameters described above can be optimally combined to reduce the energy and capital equipment required to reach a maximum level of cell disruption, thus reducing process costs. They also allow for optimizing the process on different types of biomass that possess varying ages, and degrees of lignin, a factor that affects resistance to treatment and hydrolysis and affects slurry viscosity. Enzyme loadings employed in their ratio to biomass are greatly reduced towards commercial levels when most biomass cells are disrupted and hydrolysis rates are increased. Quality of fermentable hemicellulose derived sugars produced is increased due to shorter residence times at high temperatures, in that fermentation inhibitors are reduced, and less substrate is lost to non-fermentable products. Ratios of chemicals to slurry are minimized, and utilizing nitric acid as one acid catalyst, which is compatible with stainless steel as compared with sulfuric acid, which is not, significantly reduces equipment costs, and nitric acid neutralized with ammonia into liquid stream ammonium nitrate becomes an ideal fertilizer for pumping back onto active grass production operations near a process plant. These are some of the benefits of the method.

In a preferred embodiment, wet biomass is chopped with on-the-run harvesters, then, the small-particle, chopped biomass, which is preferably less than one inch in length, is deposited into a mixing tank with added water. Alternately, to prepare dry biomass such as hay or corn stover for shearing, dry or relatively dry biomass is first reduced to a manageable size by grinding through successively smaller hammermill screens, finally through a 0.5 mm v-shaped hammermill screen such as a Pratermill by Prater Industries. In a general aspect of the process, the dry biomass is ground by conventional hammermilling to a particle size sufficiently small enough to pass through a 0.5 mm sieve. However, particle size consistency is of the greatest importance for smooth operation in the slurry cavitation machines and depending upon equipment employed in the next stage, particle sizes can be considerably larger for further processing through a slurry particle reduction system. Long rogue fibers tend to slow down the slurry's passage.

The dry-ground biomass, or the wet-chopped biomass, are mixed with water which is drawn as a slurry into a mixer-grinder-pump. A mixer-grinder-pump is a high shear, rotor-stator device capable of mixing, pumping and grinding high solid content slurries, to prepare for following stages requiring small entry level particle sizes. Preferred is an inline shear device. The inline mixer-grinder pump reduces particle size sufficiently to allow passage through a nozzle device with holes small enough to induce cavitation, preferably below 2 mm in size, but can be larger depending on overall conditions. Examples of this type of device are the HED™ manufactured and marketed by Ika Works, Inc. of Wilmington, N.C. Custom designs based upon Supraton type machines, using larger slots or round holes can produce very fine and disrupted particles from longer field chopped fibers. The inline mixer-grinder pump can have tooth and chamber type tools, and can also have nozzle tools larger than 2 mm to induce even greater shear than the tooth and chamber design tools to prepare for additional treatment under the most intense shear and cavitation conditions.

The slurry is passed through a high-shear, cavitating device with nozzle holes typically less than 2 mm in diameter at tip speeds of approximately 150 feet per second. This step can be repeated, depending upon the type of biomass being treated, specifically related to lignin content and in some cases, includes silica content. As the biomass slurry is pumped under pressure into the cavitation tools' chamber by the mixer-grinder-pump, it encounters each concentric layer of the tools in the chamber as the slurry is forced out radially. The pressure on the slurry creates the lateral radial force as it is pumped into the chamber by the mixer-grinder-pump and by the centrifugal force created by the spinning rotor. The slurry passes through the gaps between the teeth, or through the nozzle as the rotor spins past the gaps or nozzles of the stator. The result is a pulsing flow with a rapid succession of compressive and cavitational, expansion-compression forces. The lignocellulosic material in the slurry is subjected to these repeated forces, as the centrifugal force accelerates it through the gaps and holes toward the outer edge of the chamber. As the slurry moves towards the outer edge of chamber the centrifugal forces increase, thus intensifying the forces generated in the gaps. In the outer ring or rings, the slurry is forced through a gap or nozzle tool at the highest pressure within the system. The pressure is released upon the slurry containing the biomass as it exits the nozzles, and results in a violent shear upon and cavitation from without and within the cellular structures of the biomass, depending on prescribed conditions. The repeated compressive and decompressive forces create bubbles by way of cavitation in the slurry within extremely intensive energy zones. The heat and alkaline-swollen lignocellulosic coarse fibers, and most importantly, the primary cells, are ripped from the outside and blown apart from the inside by the cavitational forces, as the heated water violently vaporizes from within the primary cell structures and then just as violently re-collapses into liquid with the passing of a rotor. It is calculated that as many as half a billion such events occurs per second in a large-scale cavitation device.

In an embodiment that includes the hydrolysis, separation and concentration of hemicellulose sugars such as xylose and arabinose from biomass, followed by hydrolysis of cellulose into glucose using enzymes, cavitated biomass is treated first with acid to hydrolyze the hemicellulose while preparing the cellulosic portion of the biomass for enzymatic hydrolysis. After initial shearing and cavitation of fresh biomass is completed as above, the slurry temperature is preferably immediately increased to 205° C. by steam injection, the slurry pH is adjusted with any suitable acid at less than 1% concentration of acid wt/wt to slurry, preferably employing nitric acid, then the slurry is optionally pumped through the cavitating device one or more times during a one to three minute residence time. Alternately, the slurry is pumped through the cavitating device at a neutral pH, then the pH is adjusted immediately after as the slurry is pumped into a residence tank. Residence time is determined by the type of biomass being treated, as it relates to lignin content and when relevant, silica content, pH and corresponding ratios of acid, temperature, final yields for commercial purposes, and of great importance, residence time is related directly to minimizing or preventing production of fermentation inhibitors, including but not limited to furfurals.

The slurry and the biomass are held for a period of time sufficient to hydrolyze a high percentage of the hemicellulose, protein, fats, trace C5 sugars and some C6 sugars. Preferred is a residence time of less than 3 minutes at 205° C., or a longer time if it does not increase degradation products and if it increases yields of quality products. After sufficient residence time, the slurry is pumped out and blown down into a lower pressure tank to instantly reduce slurry temperature, and is pH neutralized with ammonia.

Some enhancement of product may be realized from the blow down step.

An alternative to the acid method above, is to employ alkaline chemicals, including but not limited to ammonium hydroxide, at 2050 C A wide range of elevated temperatures and acid or alkaline chemical concentrations are possible, in combination with tip speeds, upside and downside pressures in the cavitation devices, depending upon substrate and configurations of the high shear cavitating devices, and residence times.

Throughout this application, author and year and patents by number reference various publications, including United States patents. Full citations for the publications are listed below. The disclosures of these publications and patents in their entireties are hereby incorporated by reference into the application in order to more fully describe the state of the art to which this invention pertains. The invention has been described in an illustrative manner, and it is to be understood that the terminology that has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

1. A method for pretreating and disrupting cell structure of biomass by subjecting the biomass to high shear and cavitational decompression, by subjecting the biomass to a cavitating or shearing device having holes through which a slurry containing the biomass is pumped and subjecting the biomass to tip speeds of at least 51 feet per second.

2. The method according to claim 1, wherein said subjecting step includes subjecting the biomass to a tip speed of at least 150 feet per second.

3. The method according to claim 1, further including heating the biomass prior to cavitating or shearing to a temperature up to a boiling point of the water containing biomass without boiling the water containing the biomass.

4. The method according to claim 1, further including heating the biomass during cavitation or shearing to a temperature up to a boiling point of the water containing the biomass without boiling the biomass.

5. The method according to claim 4, further including hydrolyzing, cavitating, and shearing the biomass including heating the biomass to temperatures of at least 150° C.

6. The method according to claim 3, further including heating the biomass during cavitation or shearing to a temperature up to a boiling point of the water containing biomass without boiling the water containing the biomass.

7. The method according to claim 4 wherein said temperature subjected step further includes hydrolyzing the hemicellulose using acid loadings in the range of between 0.025-2.

8. The method according to claim 3, wherein said temperature subjecting step includes subjecting the biomass to the temperature for a range of time from between a few seconds through minutes.

9. The method according to claim 1, wherein said subjecting second stage step includes subjecting biomass ranging from 1:40 to 1:1200 by dry weight of cellulase to biomass.

10. The method according to claim 1, wherein said subjecting step includes pumping the biomass to a pressure up to 10,000 PSI.

11. The method according to claim 10, further including heating the biomass prior to and during cavitating or shearing to a temperature up to a boiling point of the water containing the biomass without boiling the water containing the biomass.

12. The method according to claim 1, wherein said subjecting step includes pumping the biomass into a nozzle.

13. The method according to claim 12, wherein said subjecting step includes passing the biomass through an exiting orifice of the nozzle.

14. The method according to claim 13, wherein said passing step includes the vaporizing stage of cavitating the slurry containing the biomass when the slurry exits the orifice of the nozzle.

15. The method according to claim 14, wherein said passing step includes condensing the water vapors surrounding the biomass in the collapsing stage of cavitation after the biomass has exited the orifice of the nozzle.

16. (canceled)

17. The method according to claim 1, wherein said subjecting step includes subjecting biomass having a solids loadings of between 0.1% and 18%.

18. The method according to claim 1, wherein said subjecting step includes subjecting the biomass to pressures up to 10,000 PSI followed by first stage of cavitational pressure release.

19. The method according to claim 1, wherein said method includes hydrolyzing the cellulosic glucose fraction of the grain and the starch glucose fraction.

20. A device for performing the method of claim 1, said device comprising: a cavitating device; and cell structure disrupting means disposed within said cavitating device for disrupting the cell structure and exposing carbohydrates within the cell, wherein said cell structure disrupting means includes holes sized between 10 millimeters and 0.25 millimeters in diameter to optimize the process.

21. The device according to claim 20, wherein said holes are sized between 5 mm and 0.5 mm in diameter.

22. (canceled)

23. (canceled)

24. (canceled)

25. (canceled)

26. (canceled)

27. (canceled)

28. (canceled)

29. (canceled)

30. (canceled)
